SEQUENCE LISTING (14 sequences)

SEQ ID NO: 1 - DNA, 24 bp, Homo sapiens
agtttcgcagacctgacatccagt

SEQ ID NO: 2 - DNA, 24 bp, Homo sapiens
ttcataactgtccttcccacggct

SEQ ID NO: 3 - DNA, 26 bp, Homo sapiens
tggaaagcgaggagttgaatggtgca

SEQ ID NO: 4 - DNA, 24 bp, Rattus norvegicus
tatcaaggtcatcccagttgccca

SEQ ID NO: 5 - DNA, 24 bp, Rattus norvegicus
atccagctgacttgactcatggct

SEQ ID NO: 6 - DNA, 25 bp, Rattus norvegicus
tctgatcaggacagcaacgggaaga

SEQ ID NO: 7 - DNA, 17 bp, Mus musculus
catcccagagctgaacg

SEQ ID NO: 8 - DNA, 18 bp, Mus musculus
ctggtcctcagtgtagcc

SEQ ID NO: 9 - DNA, 23 bp, Mus musculus
ctctctttgtcaagagtcttagc

SEQ ID NO: 10 - DNA, 25 bp, Mus musculus
agaaagattagatggcaacgagtta

SEQ ID NO: 11 - DNA, 18 bp, Mus musculus
agagtctgctgatctgcg

SEQ ID NO: 12 - DNA, 27 bp, Mus musculus
tcccatcattaaggaattcatgtcgta

SEQ ID NO: 13 - DNA, 21 bp, Mus musculus
agacacaagcattcccactat

SEQ ID NO: 14 - DNA, 17 bp, Mus musculus
caccatctcggagaccg

(19) United States
(12) Patent Application Publication
(10) Pub. No.: US 2011/0008328 A1
(43) Pub. Date: Jan. 13, 2011

(54) METHOD FOR CONTROLLING GLUCOSE UPTAKE AND INSULIN SENSITIVITY

(75) Inventors: Justin Thomas Bingham, Coronado, CA (US); Justin Guy Chapman, San Diego, CA (US); Michael Aidan North, Del Mar, CA (US); Dorothy Dee Sears, La Jolla, CA (US)

Correspondence Address: CONNOLLY BOVE LODGE & HUTZ LLP, 1875 EYE STREET, N.W., SUITE 1100, WASHINGTON, DC, 20006, US

(73) Assignees: Pfizer Inc., New York, NY (US); The Regents of the University of California, Oakland, CA (US)

(21) Appl. No.: 12/593,734

(22) PCT Filed: Apr. 17, 2008
(86) PCT No.: PCT/US2008/060619

§ 371 Date: Sep. 29, 2010

Related U.S. Application Data
(60) Provisional application No. 60/912,385, filed on Apr. 17, 2007.

(51) Int. Cl. A61K 39/395; A61K 31/7088; A61P 3/10

(52) U.S. Cl. 424/133.1; 424/130.1; 514/44 A; 424/141.1

(57) ABSTRACT

The invention provides methods for treating diabetes and related disorders, such as metabolic syndromes (which includes insulin resistance), by administering an inhibitor of osteopontin (OPN), which includes an antibody, antibody fragment, siRNA, and aptamer. Also disclosed are methods for increasing glucose uptake by cells in a subject, by administering an inhibitor of OPN.

### CROSS-REFERENCE TO RELATED APPLICATION

This application claims the benefit of U.S. Provisional Application No. 60/912,385 filed on Apr. 17, 2007, which is incorporated herein by reference in its entirety.

### FIELD OF THE INVENTION

The invention relates to methods of controlling glucose uptake by cells and insulin sensitivity in a subject by administration of an osteopontin (OPN) inhibitor. The invention further relates to the treatment of diabetes, including type 2 diabetes, and related disorders, such as metabolic syndromes (which includes insulin resistance), by administration of an OPN inhibitor. OPN inhibitors may be administered in conjunction with α-glucosidase inhibitors, insulin sensitizers, insulin secretagogues, hepatic glucose output lowering compounds, B-3 agonist, or insulin. OPN inhibitors may also be administered in conjunction with body weight reducing agents.

### BACKGROUND

Insulin resistance associated with obesity, aging, and type 2 diabetes is an increasingly prevalent disease that affects skeletal muscle, liver, adipose tissue, and immune cells. The thiazolidinedione (TZD) family of drugs are used to treat insulin resistance in a variety of pathological states, including type 2 diabetes, polycystic ovary syndrome, and “syndrome X” (Berger J P, et al., PPARs: therapeutic targets for metabolic disease. Trends in Pharmacological Sciences 26: 244-251, 2005; Bruemmer D, et al. Angiotensin II-accelerated atherosclerosis and aneurysm formation is attenuated in osteopontin-deficient mice. J Clin Invest 112: 1318-1331, 2003). TZDs are synthetic ligands for peroxisome proliferator-activated receptor gamma (PPARγ), a nuclear receptor protein that is expressed in muscle, liver, colon and macrophages and highly expressed in adipose tissue. Activated PPARγ is capable of mediating both positive and negative gene regulation and regulates gene expression through direct and indirect binding to DNA in gene promoter regions. The transcriptional activity of PPARγ varies by cell type and is modulated by other hormone signaling pathways and nuclear receptor activity. TZDs, as PPARγ ligands, regulate the expression of many genes and may enhance insulin sensitivity by altering the expression of specific genes, although the exact genes that regulate insulin action are as yet unidentified.

Adipocytes can secrete inflammatory proteins that induce macrophage activation and migration (Giorgino, F., et al. 2005. Acta Physiol Scand 183:13-30; Neels, J. G., et al. 2006. J Clin Invest 116:33-35). Obesity and insulin resistance are correlated with macrophage infiltration of adipose tissue in humans and rodent models (Bouloumie, A., et al. 2005. Curr Opin Clin Nutr Metab Care 8:347-354; Wellen, K. E., et al. 2005. J Clin Invest 115:1111-1119). Pro-inflammatory factors secreted by macrophages and adipocytes inhibit insulin sensitivity, are elevated in plasma from obese and type 2 diabetic patients, and suppress the activity of PPARγ (Grimble, R. F. 2002. Curr Opin Clin Nutr Metab Care 5:551-559). TZD-induced insulin sensitization of insulin resistant humans and mice is associated with reduced inflammatory marker gene expression and macrophages in adipose tissue (Xu, H., et al. 2003. J Clin Invest 112:1821-1830; Di Gregorio, G. B., et al. 2005. Diabetes 54:2305-2313) which may be due to TZD-activated PPARγ in macrophages and/or adipocytes. Activation of PPARγ in macrophages and adipocytes represses cytokine expression (Welch, J. S., et al. 2003, Proc Natl Acad Sci USA 100:6712-6717) and, thus, would enhance insulin sensitivity of and reduce macrophage activation and recruitment in adipose tissue. The exact genes that initiate adipose tissue inflammation and macrophage infiltration are unknown.

OPN, a secreted, extracellular matrix-associated protein, has diverse biological activities many of which make it of great interest for study in relation to insulin resistance and type 2 diabetes ((Wai, P. Y., et al. The role of osteopontin in tumor metastasis. 2004. J Surg Res 121:228-241; Bruemmer, D., et al. 2003. Angiotensin II-accelerated atherosclerosis and aneurysm formation is attenuated in osteopontin-deficient mice. J Clin Invest 112:1318-1331) and references within). For example, OPN is involved in cell migration and adhesion, macrophage activation; inflammation, tissue calcification, and matrix remodeling (Denhardt, D. T., et al. 2001, Role of osteopontin in cellular signaling and toxicant injury. Annu Rev Pharmacol Toxicol 41:723-749). OPN is over-expressed in many pathophysiological states associated with insulin resistance and type 2 diabetes, such as, in the aorta of hyperglycemic diabetics, atherosclerotic lesions, activated macrophages, steatotic hepatitis, end-stage kidney failure, and osteoporosis. To date, however, no link between OPN and the development of insulin resistance has been reported. Positive regulators of OPN expression include cytokines, e.g., IL-6, IL-1β, INF-γ, TNFα, LPS, leptin, and angiotensin II, reactive oxygen species, and hypoxia (Bruemmer, D., et al. 2003; Denhardt, D. T., et al. 2001; Ogawa, D., et al. 2005. Liver X receptor agonists inhibit cytokine-induced osteopontin expression in macrophages through interference with activator protein-1 signaling pathways. Circ Res 96:e59-67), which dampen insulin sensitivity. PPARγ and LXR ligands have been shown to antagonize OPN expression in macrophage models (Ogawa, D., et al. 2005; Oyama, Y., et al. 2002. PPARγ ligand inhibits osteopontin gene expression through interference with binding of nuclear factors to A/T-rich sequence in THP-1 cells. Circ Res 90:348-355) and in mouse aorta (Keen, H. L., et al. 2004. Gene expression profiling of potential PPARγ target genes in mouse aorta. Physiol Genomics 18:33-42). OPN is extensively and heterogeneously phosphorylated, glycosylated, and proteolysed (Christensen, B., et al. 2005. Post-translationally modified residues of native human osteopontin are located in clusters: identification of 36 phosphorylation and five O-glycosylation sites and their biological implications. Biochem J 390:285-292). Post-translational modification of OPN varies by cell type and differentially modulates its biological activity (Christensen, B., et al. 2005; Weber, G. F., et al. 2002. Phosphorylation-dependent interaction of osteopontin with its receptors regulates macrophase migration and activation. J Leukoc Biol 72:752-761). OPN binds to integrins and CD44 through which it can signal to downstream targets including phosphatidylinositol 3-kinase (PI3K), src kinase, and NFLB.

The present invention, therefore, provides methods for controlling glucose uptake and/or insulin sensitivity by administration of an OPN inhibitor. Moreover, the invention provides a novel treatment for diabetes and related disorders, that is, the administration of an OPN inhibitor.

### SUMMARY OF THE INVENTION

The present invention provides a method of controlling glucose uptake and/or insulin sensitivity by administration of an inhibitor of OPN. Moreover, the invention provides a method of treating diabetes and related diseases, such as obesity, by administering to a subject an inhibitor of OPN. Suitable inhibitors of OPN which can be employed in the methods of the invention include, but are not limited to, antibodies and antibody fragments which bind to OPN (or the receptor for OPN) and inhibit OPN binding to its receptor, OPN receptor peptide antagonists, antisense nucleic acids directed against OPN mRNA and anti-OPN ribozymes.

In another aspect, the present invention provides a method of increasing glucose uptake by a cell, by administering an OPN inhibitor. Such methods can be used, not only to treat diabetes and related diseases, but also to treat several systemic problems resulting from insufficient glucose metabolism, such as hyperglycemia.

In another aspect, the present invention provides a method of increasing insulin sensitivity in a subject having low insulin sensitivity comprising decreasing. OPN activity in the subject.

The methods of the present invention also can be performed using as targets other extracellular matrix-associated proteins, which are related in structure and activity to OPN.

### BRIEF DESCRIPTION OF THE DRAWINGS

FIGS. 1A, 1B, and 1C depict graphs showing OPN RNA levels in adipose tissue of rats and humans. (1A) depicts adipose tissue OPN RNA levels from Zucker lean (fa/+), obese (fa/fa), and pioglitazone-treated obese rats. There are 6 animals per group. (1B) depicts adipose tissue OPN RNA levels in lean patients with normal insulin sensitivity and obese patients with insulin resistance, before (black bars) and after (grey bars) pioglitazone treatment. There are 4-7 patients per group. Values are averages±standard error: * p<0.05 vs lean; #p<0.05 vs obese, before treatment. (1C) shows the correlation between adipose tissue OPN RNA levels and the rate of glucose disposal (Rd) in individual lean patients (filled symbols) and obese patients (open symbols) before pioglitazone treatment.

FIGS. 2A, 2B, and 2C depict graphs showing the characterization of insulin sensitivity. Euglycemic hyperinsulinemic clamp studies were used to calculate (2A) glucose infusion (Ginf), (2B) glucose disposal (GDR), and (2C) hepatic glucose output (HGO) in WT (black bars) and OPN KO (grey bars) mice fed normal chow or high fat diet (HFD). Values are averages±standard error. There were 7-9 animals per group were tested: * p<0.05 vs diet-matched WT, #p<0.05 vs strain-matched, normal chow.

FIGS. 3A and 3B depict adipocyte size. (3A) shows representative histological images of epidermal white adipose tissue (eWAT) from mouse groups fed normal chow (NC) or HFD. (3B) shows quantitation of inguinal white adipose tissue (iWAT) and eWAT adipocyte size. WT iWAT (black bars), WT eWAT (dark grey bars), OPN KO iWAT (light grey bars), OPN KO eWAT (white bars). Values are averages±standard error. 7 animals per group were tested: * p<0.05 vs diet-matched WT, #p<0.05 vs strain-matched, normal chow. FIG. 3C shows a correlation of fat pad mass and adipocyte size in WT (filled symbols) and OPN KO mice fed HFD (open symbols) for n=7.

FIGS. 4A and 4B depict plasma leptin. (4A) shows leptin levels that were measured by ELISA. WT (black bars) and OPN KO (gray bars) mice were fed normal chow or HFD. Values are averages±standard error. 8-10 animals per group were tested: * p<0.05 vs diet-matched WT, #p<0.05 vs strain matched, normal chow. (4B) shows correlation of plasma leptin levels with cell size in WT (filled symbols) and OPN KO (open symbols). (4C) is a correlation of plasma leptin levels with eWAT adipocyte size. (4D) shows food intake of mice on HFD was measured over 3.5 days, spanning four dark cycles. WT mice, n=5 (black bars), OPN KO mice, n=6 (hashed bars). Values are averages±standard error. * p<0.05 vs WT.

FIGS. 5A and 5B depict osteogenic and adipogenic differentiation of bone marrow stromal cells. BMSCs from the bone marrow of WT and OPN KO mice fed NC or HFD were cultured for 14 days and then subjected to osteogenic or adipogenic differentiation cocktails for the number of days shown. In (5A) osteogenic differentiation was gauged by Akp2 and OSX RNA expression relative to glycerol-3-phosphate dehydrogenase (GAPDH) expression. In (5B) adipogenic differentiation was gauged by PPARγ RNA expression. Insert bar graphs show data from WT BMSCs. Experiments were conducted in triplicate using bone marrow-derived mesenchymal stromal cells (BMSCs) isolated from 4 animals per group. Gene expression was measured by quantitative RT-PCR and data for all genes was normalized to GAPDH RNA expression. The groups are: WT mice fed NC (black bars), WT mice fed HFD (white bars), OPN KO mice fed NC (dark grey bars), OPN KO mice fed HFD (light grey bars). Values are averages±standard error: * p<0.05 vs diet-matched WT, #p<0.05 vs strain-matched, normal chow.

FIG. 6 depicts graphs showing adipose tissue cytokine levels. Cytokine protein levels were measured in eWAT lysates from WT (black bars) and OPN KO (grey bars) mice fed normal chow or HFD. Values are averages±standard error. 8-10 animals per group were tested: *p<0.05 vs diet-matched WI″, #p<0.05 vs strain-matched, normal chow. The cytokines are: IL-1β, first panel, top left; IL-12p70, second panel, left; IFNγ, third panel left; IL-6, fourth panel, bottom left; Cxcl1, first panel, top right; IL-10, second panel, right; TNFα, third panel, left; and OPN, fourth panel, bottom right.

FIGS. 7A and 7B depict insulin-stimulated Akt phosphorylation in HFD-fed mice. Phosphorylation of Ser473-Akt after 15 min. in vivo insulin stimulation was measured in tissue lysates by western blotting (7A) and ELISA (7B). ELISA data are graphed and normalized to total Akt protein: WT fed HFD (black bars), OPN KO fed HFD (grey bars). A representative western blot from each tissue is shown directly above the matching bar graph. The tissues are: Muscle—gastrocnemius muscle, iWAT—inguinal white adipose tissue, eWAT—epididymal white adipose tissue. Values are averages±standard error. 8-10 animals per group were tested: * p<0.05 vs WT.

### DETAILED DESCRIPTION

### Definitions

As used herein, the term “OPN inhibitor” or “an inhibitor of OPN” includes any agent capable of inhibiting OPN activity, including but not limited to peptides (derived from OPN or other unrelated sequences), dominant-negative protein mutants, peptidomimetics, antibodies or fragments thereof, ribozymes, antisense oligonucleotides, or other small molecules which specifically inhibit the action of OPN.

As used herein, the term “OPN activity” includes any biological activity mediated by OPN. For example, OPN is known to be involved in cell migration and adhesion, macrophage activation, inflammation, tissue calcification, and matrix remodeling (Denhardt et al., 2001). OPN is over-expressed in many pathophysiological states associated with insulin resistance and type 2 diabetes, such as, in the aorta of hyperglycemic diabetics, atherosclerotic lesions, activated macrophages, steatotic hepatitis, end-stage kidney failure, and osteoporosis.

As used herein, the term “inhibit” refers to a decrease, whether partial or whole, in function. For example, inhibition of gene transcription or expression refers to any level of downregulation of these functions, including complete elimination of these functions. Modulation of protein activity refers to any decrease in activity, including complete elimination of activity.

As used herein, the term “diabetes” includes all known forms of diabetes, including type I and type II diabetes, as described in Abel et al., Diabetes Mellitus: A Fundamental and Clinical Text (1996) pp. 530-543.

As used herein, the term “metabolic syndrome”, as used herein, unless otherwise indicated means psoriasis, diabetes mellitus, wound healing, inflammation, neurodegenerative diseases, galactosemia, maple syrup urine disease, phenylketonuria, hypersarcosinemia, thymine uraciluria, sulfinuria, isovaleric acidemia, saccharopinuria, 4-hydroxybutyric aciduria, glucose-6-phosphate dehydrogenase deficiency, and pyruvate dehydrogenase deficiency.

OPN inhibitors of the invention are typically administered to a subject in “substantially pure” form. OPN inhibitors can be substantially purified by any appropriate means known in the art.

The term “substantially pure” as used herein can refer to OPN which is substantially free of other proteins, lipids, carbohydrates, or other materials with which it is naturally associated. One skilled in the art can purify OPN using standard techniques for protein purification. The substantially pure polypeptide will yield a single major band on a non-reducing polyacrylamide gel. The purity of the OPN polypeptide can also be determined by amino-terminal amino acid sequence analysis.

As used herein, the term “modulation of OPN activity” or “modulation of OPN level” refers to a change in OPN activity or level compared to its native state. This change may be either positive (upregulation), or negative (downregulation), but for the purposes of the present invention is preferably the latter.

Cells which are targeted by the methods of the present invention, such as muscle, fat, and liver cells, include isolated cells maintained in culture as well as cells within their natural context in vivo (e.g., in fat tissue or muscle tissue, such as pectoralis, triceps, gastrocnemius, quadriceps, and iliocostal muscles, and hepatocytes).

The term “antisense nucleic acid” refers to a DNA or RNA molecule that is complementary to at least a portion of a specific mRNA molecule (Weintraub, Scientific American 262:40 (1990)). In the cell, the antisense nucleic acids hybridize to the corresponding mRNA, forming a double-stranded molecule. The antisense nucleic acids interfere with the translation of the mRNA, since the cell will not translate an mRNA that is double-stranded. Antisense oligomers of about 15 nucleotides are preferred, since they are easily synthesized and are less likely to cause problems than larger molecules when introduced into the target OPN producing cell. The use of antisense methods to inhibit the in vitro translation of genes is well known in the art (Marcus-Sakura, Anal. Biochem. 172:289 (1988)).

As used herein, a “ribozyme” is a nucleic acid molecule having nuclease activity for a specific nucleic acid sequence. A ribozyme specific for OPN mRNA, for example, would bind to and cleave specific regions of the OPN mRNA, thereby rendering it untranslatable and resulting in lack of OPN polypeptide production.

As used herein, “small interfering RNA” (siRNA) is meant an RNA molecule which decreases or silences (prevents) the expression of a gene/mRNA of its endogenous cellular counterpart. The term is understood to encompass “RNA interference” (RNAi). RNA interference (RNAi) refers to the process of sequence-specific post transcriptional gene silencing in mammals mediated by small interfering RNAs (siRNAs) (Fire et al, 1998, Nature 391, 806). The RNA interference response may feature an endonuclease complex containing an siRNA, commonly referred to as an RNA-induced silencing complex (RISC), which mediates cleavage of single-stranded RNA having sequence complementary to the antisense strand of the siRNA duplex. Cleavage of the target RNA may take place in the middle of the region complementary to the antisense strand of the siRNA duplex (Elbashir et al, 2001, Genes Dev., 15, 188). For recent information on these terms and proposed mechanisms, see Bernstein E., Denli A M., Hannon G J: The rest is silence. RNA. 2001 November; 7(11):1509-21; and Nishikura K.: A short primer on RNAi: RNA-directed RNA polymerase acts as a key catalyst. Cell. 2001 Nov. 16; 107(4):415-8.

The term “dominant-negative mutant” refers to a OPN protein which has been mutated from its natural state and which interacts with OPN or an OPN gene, thereby inhibiting its production and/or activity.

The “antibodies” of the present invention include antibodies immunoreactive with OPN polypeptides or functional fragments thereof. Antibodies which consist essentially of pooled monoclonal antibodies with different epitopic specificities, as well as distinct monoclonal antibody preparations are provided. Monoclonal antibodies are made from antigen-containing fragments of the protein by methods well known to those skilled in the art (Kohler et al, Nature 256:495 (1975)). The term “antibody” as used in this invention is meant to include intact molecules as well as fragments thereof, such as Fab and F(ab′)₂, Fv and SCA fragments which are capable of binding an epitopic determinant on OPN.

A “Fab fragment” consists of a monovalent antigen-binding fragment of an antibody molecule, and can be produced by digestion of a whole antibody molecule with the enzyme papain, to yield a fragment consisting of an intact light chain and a portion of a heavy chain.

A “Fab′ fragment” of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner.

A “(Fab′)₂” of an antibody can be obtained by treating a whole antibody molecule with the enzyme pepsin, without subsequent reduction. A (Fab′)₂ fragment is a dimer of two Fab′ fragments held together by two disulfide bonds.

An “Fv fragment” is defined as a genetically engineered fragment containing the variable region of a light chain and the variable region of a heavy chain expressed as two chains.

A “single chain antibody” (SCA) is a genetically engineered single chain molecule containing the variable region of a light chain and the variable region of a heavy chain, linked by a suitable, flexible polypeptide linker.

### OPN Inhibitors for Use in the Methods of the Invention

OPN inhibitors suitable for use in the invention include, but are not limited to, peptides, including peptides derived from OPN (e.g., mature OPN or the pro-domain of OPN) or non-OPN peptides which bind to OPN (or the receptor for OPN) and inhibit OPN binding to its receptor, OPN dominant-negative mutants, antibodies and antibody fragments which bind to OPN (or the receptor for OPN) and inhibit OPN binding to its receptor, OPN receptor peptide antagonists, antisense nucleic acids directed against OPN mRNA and anti-OPN ribozymes. Thus, OPN inhibitors can act at the message (transcription) level or at the protein (expression or activity) level.

OPN inhibitory peptides can be identified and isolated from media of cells expressing OPN using techniques known in the art for purifying peptides or proteins including ion-exchange chromatography, gel filtration chromatography, ultrafiltration, electrophoresis, and immunoaffinity purification with antibodies specific for the OPN inhibitor, or a portion thereof. In one embodiment, the media obtained from cultures of cells which express OPN are subjected to high performance liquid chromatography (HPLC). The samples obtained can then be tested for OPN inhibitory activity as described below.

Alternatively, OPN peptide inhibitors can be identified by screening fragments of OPN for inhibitory activity. Suitable assays for OPN activity can be based, e.g., on cell migration, cell adhesion, or macrophage activation. See, for example, Wai, P. Y., et al. 2004; and Bruemmer, D., et al. 2003, supra, and methods cited therein. OPN fragments can be produced by a variety of art known techniques. For example, specific oligopeptides (approximately 10-25 amino acids-long) spanning the OPN sequence can be synthesized (e.g., chemically or recombinantly) and tested for their ability to inhibit OPN, for example, using the assays described herein. The OPN peptide fragments can be synthesized using standard techniques such as those described in Bodansky, M. Principles of Peptide Synthesis, Springer Verlag, Berlin (1993) and Grant, G. A (ed.). Synthetic Peptides: A User's Guide, W. H. Freeman and Company, New York (1992). Automated peptide synthesizers are commercially available (e.g., Advanced ChemTech Model 396; Milligen/Biosearch 9600).

Alternatively, OPN fragments can be produced by digestion of native or recombinantly produced OPN by, for example, using a protease, e.g., trypsin, thermolysin, chymotrypsin, or pepsin. Computer analysis (using commercially available software, e.g. MacVector, Omega, PCGene, Molecular Simulation, Inc.) can be used to identify proteolytic cleavage sites.

OPN inhibitors used in the methods of the invention are preferably isolated. As used herein, an “isolated” or “purified” protein or biologically active peptide thereof is substantially free, of cellular material or other contaminating proteins from the cell or tissue source from which the OPN protein or peptide is derived, or substantially free from chemical precursors or other chemicals when chemically synthesized. The language “substantially free of cellular material” includes preparations of OPN protein or peptide thereof in which the protein or peptide thereof is separated from cellular components of the cells from which it is isolated or recombinantly produced. In one embodiment, the language “substantially free of cellular material” includes preparations of OPN protein or peptide thereof having less than about 30% (by dry weight) of non-OPN protein or peptide thereof (also referred to herein as a “contaminating protein”), more preferably less than about 20% of non-OPN protein or peptide thereof, still more preferably less than about 10% of non-OPN protein or peptide thereof, and most preferably less than about 5% non-OPN protein or peptide thereof. When the OPN protein or biologically active portion thereof is recombinantly produced, it is also preferably substantially free of culture medium, i.e., culture medium represents less than about 20%, more preferably less than about 10%, and most preferably less than about 5% of the volume of the protein preparation.

A two-step method can be used to produce and isolate such proteolytically cleaved OPN peptides. The first step involves enzymatic digestion of the OPN protein. OPN can be produced either as a dimer from CHO cell conditioned media or the like, as a monomer in E. coli or yeast, or isolated from cells which naturally produce OPN. Following purification of OPN monomers or dimers by, for example, HPLC chromatography, their enzymatic digestion is performed as described infra. The amino acids cleaved during the digestion depend on the specific protease used in the experiment as is known in the art. For example, if the protease of choice were trypsin, the cleavage sites would be amino acids arginine and lysine. The OPN protein can be digested using one or more of such proteases.

After the digestion, the second step involves the isolation of peptide fractions generated by the protein digestion. This can be accomplished by, for example, high resolution peptide separation as described infra. Once the fractions have been isolated, their OPN inhibitory activity can be tested for by an appropriate bioassay, as described below.

The proteolytic or synthetic OPN fragments can comprise as many amino acid residues as are necessary to inhibit, e.g., partially or completely, OPN function, and preferably comprise at least 5, 10, 15, 20, 25, 30, 35, 40, 45, 50, 55, 60, 65, 70, 75, 80, 85, 90, 95, 100 or more amino acids in length.

Other preferred peptide inhibitors of OPN are located on the surface of the OPN proteins, e.g., hydrophilic regions, as well as regions with high antigenicity or fragments with high surface probability scores can be identified using computer analysis programs well known to those of skill in the art (Hopp and Wood, (1983), Mol. Immunol., 20, 483-9, Kyte and Doolittle, (1982), J. Mol. Biol., 157, 105-32, Corrigan and Huang, (1982), Comput. Programs Biomed, 3, 163-8).

Alternatively, anti-OPN antibodies or antibody fragments can be administered directly to a subject to inhibit OPN activity. Preferred antibodies include monoclonal antibodies, including humanized, chimeric and human monoclonals or fragments thereof.

Alternatively, is also possible to immunize subjects (e.g., OPN knockout mice) with plasmids expressing OPN using DNA immunization technology, such as that disclosed in U.S. Pat. No. 5,795,872, Ricigliano et al., “DNA construct for immunization” (1998), and in U.S. Pat. No. 5,643,578, Robinson et al., “Immunization by inoculation of DNA transcription unit” (1997).

The antibody molecules directed against OPN can be isolated from the mammal (e.g., from the blood) and further purified by well known techniques, such as protein A chromatography to obtain the IgG fraction. At an appropriate time after immunization, e.g., when the anti-OPN titers are highest, antibody-producing cells can be obtained from the subject and used to prepare e.g., monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with an OPN immunogen as described above, and the culture supernatants of the resulting hybridoma cells are screened to identify a hybridoma producing a monoclonal antibody that binds OPN.

Any of the many well known protocols used for fusing lymphocytes and immortalized cell lines can be applied for the purpose of generating an anti-OPN monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations, of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind OPN, e.g., using a standard ELISA assay. The antibodies can then be tested for OPN inhibitory activity using, for example, the assays described herein.

As used herein, the term “bioassay” includes any assay designed to identify an OPN inhibitor. The assay can be an in vitro or an in vivo assay suitable for identifying whether an OPN inhibitor can inhibit one or more of the biological functions of OPN. Examples of suitable bioassays include. DNA replication assays, transcription-based assays, creatine kinase assays, assays based on the differentiation of 3T3-L1 pre-adipocytes, assays based on glucose uptake control in 3T3-L1 adipocytes, and immunological assays.

### Uses

In one embodiment, the method of the invention can be used in vivo to increase insulin sensitivity and/or glucose uptake by a cell.

In another embodiment, the method of the invention can be used to treat a disease characterized by insulin dysfunction (e.g., resistance, inactivity or deficiency) and/or insufficient glucose transport into cells. Such diseases include; but are not limited to diabetes, hyperglycemia and obesity. Other diseases include metabolic syndrome, such as insulin resistance, which may be obesity induced, polycystic ovary syndrome and aging.

In another embodiment, the method of the invention can be used to treat a disease characterized by insulin dysfunction (e.g., resistance, inactivity or deficiency) and/or insufficient glucose transport into cells. Such diseases include, but are not limited to diabetes, hyperglycemia and obesity.

In another embodiment, the method of the invention can be used to create a novel in vitro model, in which OPN is utilized to examine glucose uptake or glucose metabolism in adipocytes. OPN, which is specifically expressed in muscle and fat in vivo, inhibits 3T3-L1 adipocyte differentiation by directly or indirectly suppressing the expression of adipocyte-specific genes. OPN can, therefore, be used as a prototype regulator of these genes in the 3T3-L1 cell system. This system can be a model for understanding the role of OPN on the regulation of adipocyte-specific gene expression and protein activity of molecules such as, but not limited to, transcription factors, signal transduction proteins, leptin, fatty acid binding protein, fatty acid synthase, peroxisome proliferator-activated receptors, uncoupling proteins 1 and 2, and molecules that are activated, inactivated, or modified by the actions of OPN.

In another embodiment, the OPN inhibitor can be SiRNA. During recent years, RNAi has emerged as one of the most efficient methods for inactivation of genes (Nature Reviews, 2002, v. 3, p. 737-47; Nature, 2002, v. 418, p. 244-51). As a method, it is based on the ability of dsRNA species to enter a specific protein complex, where it is then targeted to the complementary cellular RNA and specifically degrades it. In more detail, dsRNAs are digested into short (17-29 bp) inhibitory RNAs (siRNAs) by type III RNAses (DICER, Drosha, etc) (Nature, 2001, v. 409, p. 363-6; Nature, 2003, 425, p. 415-9). These fragments and complementary mRNA are recognized by the specific RISC protein complex. The whole process is culminated by endonuclease cleavage of target mRNA (Nature reviews, 2002, v. 3, p. 737-47; Curr Opin Mol. Ther. 2003 June; 5(3):217-24). For disclosure on how to design and prepare siRNA to known genes see for example Chalk A M, Wahlestedt C, Sonnhammer E L. Improved and automated prediction of effective siRNA Biochem. Biophys. Res. Commun. 2004 Jun. 18; 319(1):264-74; Sioud M., Leirdal M., Potential design rules and enzymatic synthesis of siRNAs, Methods Mol. Biol. 2004; 252:457-69; Levenkova N, Gu Q, Rux J J.: Gene specific siRNA selector Bioinformatics. 2004 February 12:20(3):430-2. and Ui-Tei K, Naito Y, Takahashi F, Haraguchi T, Ohki-Hamazaki H, Juni A. Ueda R, Saigo K., Guidelines for the selection of highly effective siRNA sequences for mammalian and chick RNA interference Nucleic Acids Res. 2004 Feb. 9; 32(3):936-48. See also Liu Y, Braasch D A, Nulf C J, Corey D R. Efficient and isoform-selective inhibition of cellular gene expression by peptide nucleic acids Biochemistry, 2004 Feb. 24; 43(7):1921-7. See also PCT publications WO 2004/015107 (Atugen) and WO 02/44321 (Tuschl et al), and also Chiu Y L, Rana T M. siRNA function in RNAi: a chemical modification analysis, RNA 2003 September; 9(9):1034-48 and U.S. Pat. Nos. 5,898,031 and 6,107,094 (Crooke) for production of modified/more stable siRNAs.

DNA-based vectors capable of generating siRNA within cells have been developed. The method generally involves transcription of short hairpin RNAs that are efficiently processed to form siRNAs within cells. Paddison et al. PNAS 2002, 99:1443-1448; Paddison et al. Genes & Dev 2002, 16:948-958; Sui et al. PNAS 2002, 8:5515-5520; and Brummelkamp et al. Science 2002, 296:550-553. These reports describe methods to generate siRNAs capable of specifically targeting numerous endogenously and exogenously expressed genes.

For delivery of siRNAs, see, for example, Shen et at (FEBS letters 539:111-114 (2003)), Xia et al., Nature Biotechnology 20: 1006-1010 (2002), Reich et al., Molecular Vision 9: 210-216 (2003), Sorensen et al. (J. Mol. Biol. 327:761-766 (2003), Lewis et al., Nature Genetics 32: 107-108 (2002) and Simeoni et al., Nucleic Acids Research 31, 11:2717-2724 (2003). siRNA has recently been successfully used for inhibition in primates; for further details see Tolentino et al., Retina 24(1) February 2004 pp. 132-138.

Other uses for the methods of the invention will be apparent to one of ordinary skill in the art from the following Examples and Claims.

### Administration of OPN Inhibitors in Pharmaceutical Compositions

OPN inhibitors used in the methods of the present invention are generally administered to a subject in the form of a suitable pharmaceutical composition. Such compositions typically contain the inhibitor and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the OPN inhibitor, use thereof in the compositions is contemplated. Supplementary active compounds can also be incorporated into the compositions.

A pharmaceutical composition of the invention is formulated to be compatible with its intended route of administration. Examples of suitable routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the OPN inhibitor in the required amount in an appropriate solvent with one or a combination of ingredients enumerated above, as required, followed by filtered sterilization. Generally, dispersions are prepared by incorporating the OPN inhibitor into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the OPN inhibitor can be incorporated with excipients and used in the form of tablets, troches, or capsules, oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The OPN inhibitor can also be prepared in the form of suppositories (e.g., with conventional suppository bases such as cocoa butter and other glycerides) or retention enemas for rectal, delivery.

In one embodiment, the OPN inhibitors are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀ OPN inhibitors which exhibit large therapeutic indices are preferred. While OPN inhibitors that exhibit toxic side effects may be used, care should be taken to design a delivery system that targets such OPN inhibitors to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any OPN inhibitor used in the method of the invention, the therapeutically effective dose can be estimated initially from cell culture assays. A dose may be formulated in animal models to achieve a circulating plasma concentration range that includes the IC₅₀ (i.e., the concentration of the test OPN inhibitor which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

The OPN inhibitors of the present invention, e.g., the anti-sense oligonucleotide inhibitors, can further be inserted into vectors and used in gene therapy. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Vectors suitable for use in gene therapy are known in the art. For example, adenovirus-derived vectors can be used. The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they are not capable of infecting nondividing cells. Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al. (1979) Cell 16:683; Berkner et al., supra; and Graham et al. in Methods in Molecular Biology, E. J. Murray, Ed. (Humana, Clifton; N.J., 1991) vol. 7. pp. 109-127). Expression of the gene of interest comprised in the nucleic acid molecule can be under control of, for example, the E1A promoter, the major late promoter (MLP) and associated leader sequences, the E3 promoter, or exogenously added promoter sequences.

Yet another viral vector system useful for delivery of the OPN inhibitors of the invention is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). Adeno-associated viruses exhibit a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as few as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into T cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790). Other viral vector systems that may be useful for delivery of the OPN inhibitors of the invention are derived from herpes virus, vaccinia virus, and several RNA viruses.

Insulin resistance (IR) is manifested in muscle, adipose tissue (AT), and liver and is associated with inflammation in AT. A subject has IR if the response to insulin is half the response in a normal subject. IR can be measured by use of the hyperinsulinemic euglycemic clamp, the Reaven modified insulin suppression test, by the homeostatic model assessment, or the quantitative insulin sensitivity check index. The present invention is based, in part, on the discovery that mRNA expression of the proinflammatory protein OPN was elevated in the AT of obese, insulin resistant humans and rats and was normalized after thiazolidinedione treatment in both species. The present inventors studied the role of OPN in IR using OPN knockout mice (OPN KOs) and a two week high fat diet (HFD) model of IR. OPN KOs were completely protected from the severe HFD-induced changes in insulin stimulated glucose disposal rate and hepatic glucose output that were observed in wild type mice (WTs). HFD did not alter body weight, AT macrophage infiltration, or plasma free fatty acids and cytokines. HFD-induced hyperleptinemia, AT cytokine secretion, and adipocyte hypertrophy were blunted or absent in OPN KOs vs. WTs. Muscle and eWAT insulin-stimulated Akt phosphorylation was greater in HFD-fed OPN KOs vs. WTs. OPN KO bone marrow stromal cells were more osteogenic and less adipogenic than WT cells. Both differentiation pathways were affected by HFD in WT cells. These OPN KO phenotypes correspond with protection from IR. OPN is a key component of diet-induced IR, before obesity and AT macrophage infiltration occur. It was further discovered as part of the present invention that OPN is involved in cell migration, macrophage activation, inflammation and extracellular matrix remodeling. These biological activities are all activated in the adipose tissue of insulin resistant animal and human models.

### EXAMPLES

The following examples are presented only as exemplary ways of practicing the invention. These examples are intended to illustrate but not limit the invention. While they are typical of those that might be used, other procedures known to those skilled in the art may alternatively be used.

### Example 1

### Gene Expression: Identification of OPN as a Potential Target of Intervention for Insulin Resistance

As insulin resistance is associated with altered gene expression, Affymetrix was used to profile global gene expression to identify genes that are differentially regulated in the adipose tissue of insulin resistant human patients (n=34) compared to lean insulin sensitive patients (n=6) and normalized by treatment with insulin-sensitizing thiazolidinediones (TZDs). More than 300 genes were identified displaying differential expression between the insulin resistant and insulin sensitive patients. A similar approach was performed utilizing the insulin resistant rodent fa/fa model. Differential gene expression was measured in adipose tissue of Zucker lean (fa/f), Zucker obese (fa/fa), and 14-day TZD-treated obese rats. Global gene expression profiling of adipose tissue in this model identified more than 100 genes that were differentially regulated in the insulin resistant fa/fa rats as compared to their lean controls. Using the overlap of the human and rat expression profiling study, the inventors were able to identify 4 genes that displayed differential expression in adipose tissue of insulin resistant humans and rats and which were normalized by TZD treatment as potential targets for intervention. OPN, ALCAM, GLIPR1, and S100 A4 were identified as candidate genes. As OPN is a known secreted protein and the statistically most highly significant transcript identified, to confirm the results the inventors used quantitative RT-PCR to compare OPN expression in adipose tissue from lean and obese humans and Zucker rats, before and after treatment with the insulin-sensitizing TZD, pioglitazone.

### Example 2

### Insulin Sensitivity

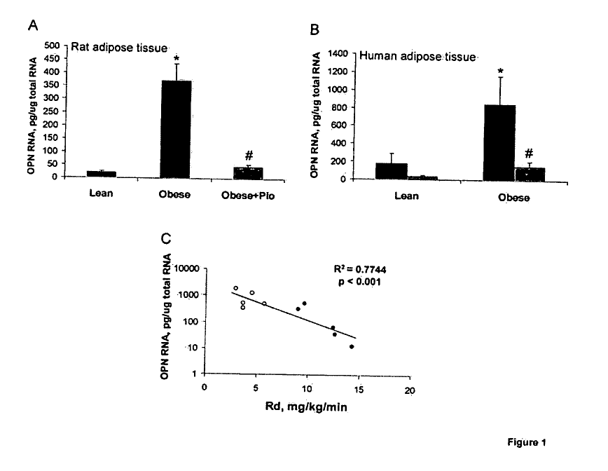

Peripheral insulin sensitivity (measured as the rate of glucose disposal, Rd) of the rat and human groups was measured using the hyperinsulinemic euglycemic clamp procedure. Clinical characteristics of the rats and humans are detailed in Table 1. OPN expression in adipose tissue was elevated in obese, insulin resistant rats (17-fold) and humans (4.6-fold) compared to insulin sensitive lean controls. Pioglitazone treatment increased the insulin sensitivity of the obese rats and humans (Table 1) and normalized OPN expression in the adipose tissue of both species (FIGS. 1A and 1B). In fact, when the pre-treatment patient data was combined, there was a significant correlation between adipose tissue OPN RNA levels and Rd (FIG. 1C).

TABLE 1
Clinical Characteristics

| Zucker Rats | Lean, no Rx | Obese, no Rx | Obese, post-Rx |
|---|---|---|---|
| All male, 9 weeks of age, n = 7 per | | | |
| group | | | |
| Weight (g) | 236 ± 6 | 363 ± 17 * | 387 ± 14 * |
| Fasting plasma glucose (mg/dL) | 117.4 ± 4.4 | 137.1 ± 8.6 * | 124.8 ± 4.0 |
| Glucose disposal, Rd (mg/kg/min) | 38.4 ± 2.9 | 18.0 ± 2.3 * | 30.6 ± 2.9 ^{#} |
| Fasting plasma insulin (pmol/L) | 1.3 ± 0.2 | 24.7 ± 5.8 * | 4.8 ± 1.4 *^{#} |
| Fasting plasma free fatty acids | 1.59 ± 1.4 | 2.89 ± 0.94 | 0.69 ± 0.29 *^{#} |
| (mmol/L) | | | |
| Fasting plasma triglyceride | 44.7 ± 5.1 | 276.7 ± 54.4 * | 108.0 ± 16.1 *^{#} |
| (mg/dL) | | | |

| Human Patients | Lean, pre-Rx | Lean, post-Rx | Obese, pre-Rx | Obese, post-Rx |
|---|---|---|---|---|
| Gender (male/female) | 4/1 | | 5/1 | |
| Age (years) | 45.2 ± 8.7 | | 53.8 ± 2.1 | |
| BMI (kg/m²) | 23.1 ± 1.0 | 23.6 ± 1.2 | 36.3 ± 2.3 * | 37.2 ± 3.2 * |
| Fasting plasma glucose | 90.4 ± 3.6 | 87.6 ± 3.1 | 97.7 ± 4.5 | 91.7 ± 2.5 |
| (mg/dL) | | | | |
| Glucose disposal, Rd | 11.7 ± 1.1 | 10.6 ± 0.8 | 4.2 ± 0.4 * | 5.9 ± 0.5 *^{#} |
| (mg/kg/min) | | | | |
| Fasting plasma insulin | 10.3 ± 1.4 | 10.1 ± 2.2 | 22.1 ± 3.9 * | 13.6 ± 1.6 ^{#} |
| (pmol/L) | | | | |
| Fasting plasma free | 423 ± 90 | 414 ± 90 | 441 ± 43 | 434 ± 59 |
| fatty acids (mmol/L) | | | | |
| Fasting plasma | 88.6 ± 3.7 | 76.0 ± 9.2 | 182.3 ± 42.2 | 137.7 ± 7.2 * |
| triglyceride (mg/dL) | | | | |

Table 1 note:
Data are averages ± standard error. Rats were treated with pioglitazone for 3 weeks, humans were treated with pioglitazone for 3 months.
* p < 0.05 vs. lean subjects.
^{#} p < 0.05 vs. pre-Rx subjects.
Rx - pioglitazone treatment.

### Example 3

### OPN KO Mice are Protected from Diet-Induced Insulin Resistance

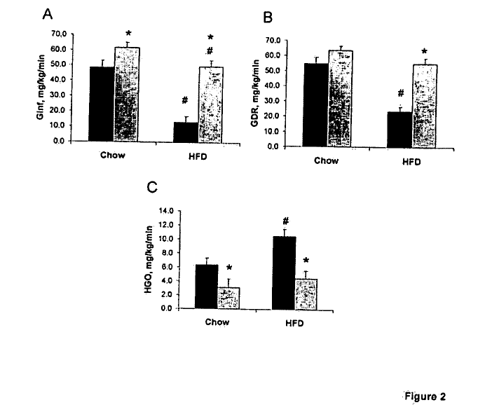

The in vivo effects of whole-body OPN gene knockout in early onset, diet-induced insulin resistance were evaluated using C57BL/6 WT and strain-matched OPN KO mice. Euglycemic hyperinsulinemic clamp studies were conducted on WT and OPN KO mice fed NC or HFD for two weeks and significant differences in clamp data from the mouse strains fed both diets were observed (FIG. 2A-C). The glucose infusion rate (Ginf) during the clamp was 27% greater in the OPN KO mice fed NC compared to WT mice. The glucose disposal rate (GDR) during the clamp in the OPN KO mice fed NC tended to be greater as well. The hepatic glucose output rate (HGO) during the clamp of the OPN KO mice fed NC was 52% lower compared to WT mice. In addition, the OPN KO mice were protected from the severe HFD-induced decrease in Ginf (73%) and GDR (57%) and increase in HGO (66%) that we observed in WT mice. These data indicate that the absence of OPN leads to improved hepatic insulin sensitivity when mice are fed NC and protection from hepatic and skeletal muscle insulin resistance when mice are fed HFD.

Aside from differences in insulin sensitivity detected by euglycemic hyperinsulinemic clamp, many similarities between HFD-induced changes in WT and OPN KO mice were observed. Also, the two week HFD model of insulin resistance presents without many of the secondary abnormalities observed in longer HFD models. Total body weight was not different between the mouse strains and was unchanged by HFD (Table 2). Epididymal white adipose tissue (eWAT) fat pad weights increased similarly in all animals on HFD and, as a percentage of total body weight, were not different between the strains (Table 2). Fasting plasma insulin levels tended to be higher in the NC- and HFD-fed WT mice compared to the OPN KO mouse groups, but the differences were not significant by ANOVA analysis (p=0.068) (Table 2). Fasting plasma insulin levels tended to be higher in the NC- and HFD-fed WT mice compared to the OPN KO mouse groups, but the differences were not significant by ANOVA analysis (p=0.068) (Table 2).

TABLE 2
Mouse strain characteristics

| | WT NC | | WT HFD | | OPN KO NC | | KO HFD | |
|---|---|---|---|---|---|---|---|---|
| Whole body weight, g | 27.5 | (0.7) | 29.3 | (0.9) | 27.2 | (0.8) | 28.7 | (0.7) |
| Epididymal fat pad weight, g | 0.24 | (0.02) | 0.71 | (0.08) # | 0.26 | (0.02) | 0.60 | (0.05) # |
| Epididymal fat pad mass, % body weight | 0.87 | (0.08) | 2.37 | (0.25) # | 0.95 | (0.08) | 2.13 | (0.14) # |
| Fasting plasma glucose, mg/dL | 162 | (6) | 172 | (11) | 177 | (9) | 203 | (8) * |
| Fasting plasma insulin, ng/mL | 1.65 | (0.44) | 1.89 | (0.78) | 0.62 | (0.17) | 0.61 | (0.05) |

Data are averages ± standard error. 7-10 mice per group.
* p < 0.05 vs diet-matched WT,
# p < 0.05 vs strain-matched NC.

Fasting plasma glucose levels were slightly elevated only in the OPN KO mice fed HFD. The present inventors measured the levels of other plasma components in the four animal groups including OPN, adipokines, cytokines, chemokines, and lipids (Table 3). With the exception of total cholesterol, none of the plasma components in Table 3 were elevated in either the WT or OPN KO mice fed HFD. Total plasma cholesterol was elevated in both mouse strains fed HFD but was slightly lower in the OPN KO mice.

TABLE 3
Tissue and plasma components

| | WT NC | | WT HFD | | KO NC | | KO HFD | |
|---|---|---|---|---|---|---|---|---|
| Plasma Cytokines, | | | | | | | | |
| etc. | | | | | | | | |
| OPN (ng/mL) | 5108 | (382.4) | 5772 | (568.5) | N.D. | | N.D. | |
| ACRP30 (ug/mL) | 22.7 | (1.7) | 27.9 | (4.0) | 25.6 | (3.2) | 35.6 | (6.3) |
| MCP-1 (pg/mL) | 42.63 | (1.1) | 43.87 | (14.2) | 73.49 | (12.8) | 64.0 | (9.6) |
| IL-1β (pg/mL) | 43.1 | (5.1) | 22.4 | (6.0) # | 36.0 | (2.8) | 22.2 | (2.0) |
| IL-12p70 (pg/mL) | 59.5 | (7.3) | 39.4 | (10.4) | 50.6 | (2.6) | 37.8 | (4.6) |
| IFNγ (pg/mL) | 14.1 | (1.7) | 11.0 | (3.4) | 12.9 | (0.8) | 9.8 | (1.3) |
| IL-6 (pg/mL) | 218.5 | (16.5) | 179.7 | (48.2) | 187.1 | (11.1) | 167.0 | (24.8) |
| KC (CXCL-1) (pg/mL) | 84.4 | (5.7) | 67.2 | (9.9) | 93.5 | (14.9) | 63.5 | (3.7) |
| IL-10 (pg/mL) | 130.2 | (1.1) | 129.9 | (1.6) | 135.9 | (1.9) | 129.4 | (1.1) |
| TNFα (pg/mL) | 38.7 | (3.5) | 31.5 | (4.1) | 41.7 | (3.2) | 31.7 | (1.2) |
| Resistin (pg/mL) | 826.9 | (66.5) | 762.5 | (92.7) | 1067.0 | (115.5) | 788.8 | (38.4) |
| Tissue and plasma | | | | | | | | |
| Lipids | | | | | | | | |
| Total plasma | 78.4 | (2.2) | 147.8 | (20.0) # | 78.9 | (2.5) | 127.0 | (3.6) *# |
| cholesterol (mg/dL) | | | | | | | | |
| Plasma triglycerides | 31.0 | (3.5) | 28.8 | (4.2) | 39.2 | (6.5) | 24.6 | (1.6) # |
| (mg/dL) | | | | | | | | |
| Plasma free fatty acids | 0.433 | (0.025) | 0.454 | (0.099) | 0.418 | (0.024) | 0.370 | (0.027) |
| (mmol/L) | | | | | | | | |
| Skeletal muscle | 15.3 | (1.0) | 15.7 | (2.9) | 12.5 | (0.8) * | 14.7 | (0.9) |
| triglycerides (mg/dL) | | | | | | | | |
| Liver triglycerides | 6.7 | (1.1) | 9.7 | (1.3) | 7.2 | (1.0) | 8.5 | (1.2) |
| (mg/dL) | | | | | | | | |

Table 3 note:
n = 7-10 mice per group.
* p < 0.05 vs diet-matched WT,
# p < 0.05 vs strain-matched NC.
ND means no data.

### Example 4

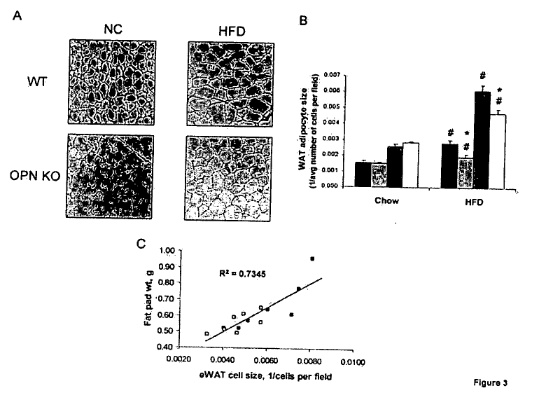

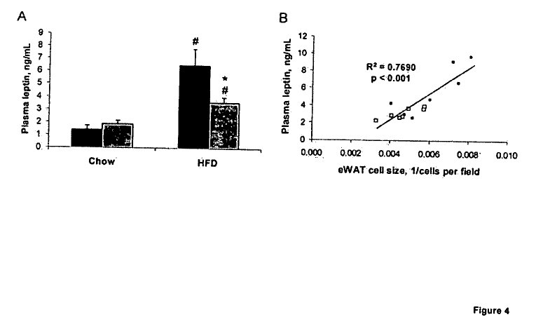

Differential histology of WAT and plasma leptin levels in OPN KO mice. The histological sections of the eWAT (a visceral adipose depot) and inguinal adipose tissue (iWAT, a subcutaneous adipose depot) were examined and it was found that HFD-induced adipocyte hypertrophy in eWAT and iWAT from WT mice was blunted 23% and 30%, respectively, in the OPN KO mice (FIG. 3). Given the comparable eWAT fat pad mass in the two strains fed HFD, there may be hyperplasia of smaller adipocytes in the OPN KO mice. There were no gross differences observed in extracellular matrix or other non-adipocyte cell structures when comparing the OPN KO and WT WAT histological sections. The eWAT and iWAT sections were also examined for the presence of adipose tissue macrophages using immunohistochemistry to detect the macrophage-specific marker Mac-2. Mac-2 staining has previously been shown to increase in adipose tissue from obese mice and humans (Cinti, S., et al. 2005. J Lipid Res 46:2347-2355). No crown-like structures or differences in the number of Mac-2 stained cells were detected when comparing sections from NC-fed and HFD-fed mice and there were no differences between the strains, suggesting that macrophage infiltration into adipose did not play a role in insulin resistance after two week HFD. Plasma leptin levels correlate with fat mass in multiple species models of obesity (Fruhbeck, G. 2006, Biochem J 393:7-20). Thus, plasma leptin levels were measured in the four mouse groups and it was, found that HFD-induced increase in plasma leptin in WT mice (4.6-fold) was blunted 45% in OPN KO mice (FIG. 4A). Although the average fat pad mass was not significantly different between the HFD-fed WT and OPN KO mice (Table 2 and FIG. 4B), plasma leptin levels significantly correlate with eWAT adipocyte size in individual mice (FIG. 4C).

### Example 5

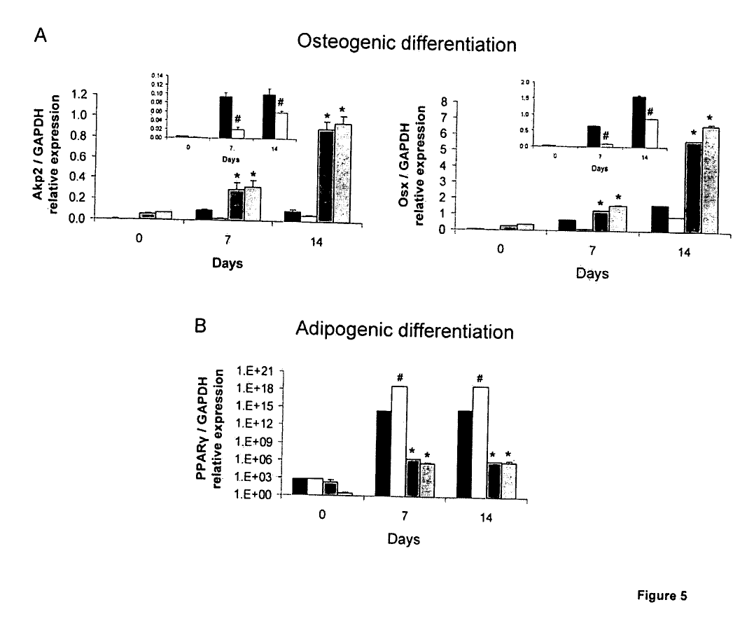

Analysis of tissue components associated with insulin resistance. The present inventors studied tissue components in the mouse groups to identify strain and/or diet-dependent differences that relate to insulin resistance. In several models of diet-induced insulin resistance, triglyceride accumulation is observed in muscle and liver. Table 3 shows the tissue triglyceride levels that were measured in muscle and liver. Triglyceride levels were significantly lower in skeletal muscle from OPN KO mice fed NC compared to WT mice. However, there was no difference in muscle triglyceride between the strains fed HFD, neither was there a significant increase in triglyceride as a result of HFD. Triglyceride levels in liver tended to increase in the HFD-fed WT and OPN KO mice, but this trend was not significant and was not different between the strains. As adipose tissue is the location of local inflammation in obesity, the cytokine protein levels were examined in this tissue. Although HFD did not increase plasma cytokine levels in either strain, elevated levels of IL-1β, IL-12p70, IFNγ, IL-6, and IL-10 were observed in eWAT lysates from WT mice (FIG. 5). Cxcl1 (KC) and TNFα levels also tended to increase after HFD in WT mice but this increase did not reach statistical significance. HFD-fed OPN KO mice were completely protected from increases in eWAT lysate IL-1β, IL-12p70, IFNγ, IL-6, IL-10, Cxcl1 and TNFα levels.

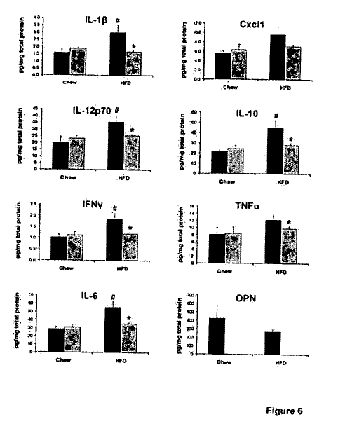

Differential activation of Akt phosphorylation after HFD. In order to examine the effects of OPN KO on insulin signal transduction in mice fed HFD, the present inventors acutely insulin-stimulated the groups by intraperitoneal injection of 0.85 mU/kg insulin and harvested tissue after 15 min. ELISA quantitation of insulin-simulated muscle and adipose Akt phosphorylation in these studies is shown in FIG. 6. Insulin-stimulated Akt phosphorylation in muscle was 58% greater in OPN KO mice compared to WT mice. Insulin-stimulated Akt phosphorylation in iWAT was not different between the strains but was 73% greater in eWAT from OPN KO mice compared to WT mice. In parallel, the present inventors analyzed the tissue lysates by SDS-PAGE and western blotting. As with the ELISA results, insulin stimulated Akt phosphorylation was significantly greater in the muscle and eWAT from OPN KO mice compared to WT mice.

### Example 6

Differentiation of bone marrow stromal cells. Bone marrow-derived mesenchymal stromal cells (BMSCs) are multi-potent and can differentiate along osteogenic; adipogenic and chondrogentic pathways (Gimble, J. M., et al. 2006, J Cell Biochem 98:251-266). A study was done to analyze whether the absence of OPN expression and/or diet affected the propensity of BMSCs to differentiate through two of these pathways.

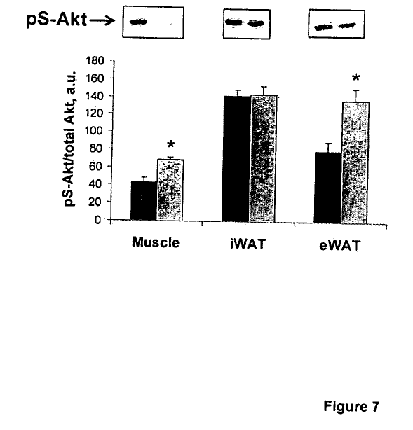

BMSCs were harvested from four mouse groups and they were subjected to both an osteogenic protocol and an adipogenic protocol. Progression through each differentiation program was measured by expression of the osteoblast genes alkaline phosphatase (Akp2) and osterix (Osx) and the adipocyte gene PPARγ (FIG. 7). Expression of the Akp2 and Osx during osteogenic differentiation was significantly greater in OPN KO BMSCs than in WT BMSCs (FIG. 7A). HFD significantly blunted WT BMSC osteogenic differentiation but had no effect on OPN KO osteogenic differentiation. Expression of PPARγ during the adipogenic protocol increased 1000-fold in OPN KO BMSCs but increased nine orders of magnitude more in WT BMSCs (FIG. 7B). HFD significantly enhanced WT BMSC adipogenic differentiation (1000-fold more PPARγ expression) but had no effect on OPN KO adipogenic differentiation. It is notable that the two week HFD had such an effect on WT BMSC osteogenic and adipogenic differentiation after ex vivo culture for more than four weeks. It is likely that HFD programs a differentiation potential bias in the BMSCs, in vivo. Together, these data suggest that OPN deletion enhances osteogenic and inhibits adipogenic differentiation of BMSCs and, conversely, HFD inhibits osteogenic and enhances adipogenic differentiation of BMSCs.

Insulin resistance is associated with chronic low grade inflammation in adipose tissue. Although the known biological roles of OPN include regulating inflammation, a role for OPN in regulating adipose tissue biology and/or whole body metabolism has only recently been described (Nomiyama T, et al., Osteopontin mediates obesity-induced adipose tissue macrophage infiltration and insulin resistance in mice. J Clin Invest 117: 2877-2888, 2007). The present results show that OPN RNA expression was elevated in adipose tissue of obese insulin resistant rats and humans, was correlated with Rd in humans, and was normalized after TZD treatment. In global transcriptional profiling analyses, Xu, et al. found that OPN is one of 50 inflammatory genes over-expressed in WAT from five mouse models of obesity (Xu H, et al., 2003). OPN over-expression in adipose tissue from obese, insulin resistant subjects may be attributable to adipocytes and/or stromal vascular cells in response to activation by leptin, cytokines, or lipid modulators. OPN is expressed in many cell types (Denhardt D T et al., 2001, supra) and is also expressed in 3T3-L1 adipocytes (Ross S E, et al., Microarray analyses during adipogenesis: understanding the effects of Wnt signaling on adipogenesis and the roles of liver X receptor alpha in adipocyte metabolism. Mol Cell Biol 22: 5989-5999, 2002; and the inventor's data). TZD-mediated insulin sensitization is associated with decreased adipose tissue inflammation (Di Gregorio G B, et al., 2005). OPN expression is down-regulated by PPARγ and LXR ligands in macrophage models (Ogawa D, et al., 2005; Oyama Y, et al., 2002; Oyama Y, et al., Troglitazone, a PPARgamma ligand, inhibits osteopontin gene expression in human monocytes/macrophage THP-1 cells. J Atheroscler Thromb 7: 77-82, 2000). The OPN KO data disclosed above suggest that normalization of OPN expression in adipose tissue may play a key role in TZD-mediated sensitization by reducing inflammation.

The present inventors investigated the role of OPN in the development of insulin resistance in an early-onset, two week HFD model. Notably, the two week HFD was not associated with detectable changes in body weight, liver or skeletal muscle triglyceride, macrophage infiltration into adipose tissue, plasma FFA or triglyceride, or plasma inflammatory markers (with the exception of leptin). The phenotypes of early onset high fat feeding disclosed herein are similar to those observed by Park, et al. in their timecourse study of HFD-induced insulin resistance (Park S Y, et al., Unraveling the temporal pattern of diet-induced insulin resistance in individual organs and cardiac dysfunction in C57BL/6 mice. Diabetes 54: 3530-3540, 2005). After two weeks, HFD induced severe hepatic and skeletal muscle insulin resistance in WT mice, and hyperinsulinemia, hypercholesterolemia, and fat pad expansion in both mouse strains. OPN KO mice were protected from HFD-induced hepatic and skeletal muscle insulin resistance and had greater hepatic insulin sensitivity than WT mice when fed NC. OPN KO mice also exhibited significantly enhanced insulin-stimulated skeletal muscle and eWAT Akt phosphorylation after HFD, compared to WT mice, which corresponds with their enhanced insulin sensitivity. Interestingly, insulin-stimulated iWAT Akt phosphorylation was not different between the strains fed HFD, which indicates that this subcutaneous adipose depot was not insulin resistant, unlike the eWAT depot.

HFD increased plasma leptin levels in WT mice and this increase was blunted in OPN KO mice. The present inventors observed similar differences in the effect of HFD on leptin RNA expression in adipose tissue from these strains. In addition, the plasma leptin levels were correlated with eWAT adipocyte size in both WT and OPN KO mice. In several obesity models, leptin levels are elevated, reflect adipose mass, and correlate with adipocyte size in humans (Frederich R C, et al., Expression of ob mRNA and its encoded protein in rodents. Impact of nutrition and obesity. J Clin Invest 96: 1658-1663, 1995; Fruhbeck, 2006; Lofgren P, et al., Long-term prospective and controlled studies demonstrate adipose tissue hypercellularity and relative leptin deficiency in the post-obese state. J Clin Endocrinol Metab 90: 6207-6213, 2005). TZDs and weight loss both reduce plasma leptin levels and adipocyte size (Lofgren et al., 2005, supra; Yamauchi T, et al., The mechanisms by which both heterozygous peroxisome proliferator-activated receptor gamma (PPARgamma) deficiency and PPARgamma agonist improve insulin resistance. J Biol Chem 276: 41245-41254, 2001). Leptin has inflammatory activity and can activate secretion of proinflammatory cytokines (Fruhbeck, 2006, supra; Sanchez-Margalet V, et al., Role of leptin as an immunomodulator of blood mononuclear cells: mechanisms of action. Clin Exp Immunol 133: 11-19, 2003). Leptin induces Ser-318 phosphorylation of IRS1 in lymphocytes and skeletal muscle, inhibiting insulin signal transduction in muscle (Hennige A M, et al., Leptin down-regulates insulin action through phosphorylation of serine-318 in insulin receptor substrate 1. FASEB J 20: 1206-1208, 2006). Cultured hepatocytes from db/db mice increase mRNA and protein expression of OPN after treatment with leptin (Sahai A, et al., Obese and diabetic db/db mice develop marked liver fibrosis in a model of nonalcoholic steatohepatitis: role of short-form leptin receptors and osteopontin. Am J Physiol Gastrointest Liver Physiol 287: G1035-1043, 2004). Thus, higher leptin levels in the plasma and, presumably, adipose tissue of HFD-fed WT mice may contribute to the multi-tissue insulin resistance we observe in these mice compared to OPN KO mice. Further studies of the mechanistic connection between OPN and leptin in various tissues are ongoing.

Adipocytes from OPN KO mice were significantly less hypertrophic after HFD than the adipocytes from WT mice. Decreased HFD-induced hypertrophy of OPN KO adipocytes may be related to the decreased adipogenic potential of OPN KO BMSCs compared to WT BMSCs and is discussed in more detail below. In addition to secreting more leptin, larger adipocytes are less insulin sensitive, secrete more inflammatory cytokines and FFAs, and produce more reactive oxygen species and than smaller adipocytes (Pausova Z. From big fat cells to high blood pressure: a pathway to obesity-associated hypertension. Curr Opin Nephrol Hypertens 15: 173-178, 2006). The present observations show both decreased insulin sensitivity and increased cytokine secretion in eWAT from HFD-fed WT mice compared to OPN KO mice. Increased cytokine secretion could be attributable to resident macrophages, adipocytes, or other cell types in eWAT such as endothelial cells and preadipocytes. Leptin and OPN both induce inflammatory cytokine secretion (Denhardt, 2001, supra; Fruhbeck, 2006, supra) and can be mediators of the increased cytokine secretion that were observed in the HFD-fed WT eWAT. No changes in eWAT OPN protein from the WT mice were observed after HFD, although there may be a HFD-induced shift in OPN's biological activity mediated by differential glycosylation, phosphorylation, and/or proteolysis, as has been shown in other systems (Christensen B, et al., 2005; Weber G F, et al., 2002). Based on the human and rat expression data disclosed herein and on the data of Xu et al. (2003), it appears that OPN expression would increase after longer HFD.

Both in vivo and in vitro studies have demonstrated an inverse relationship between the differentiation of BMSCs into osteoblasts and adipocytes (Gimble J M, et al., 2006, supra; Rosen C J et al., Mechanisms of disease: is osteoporosis the obesity of bone? Nat Clin Pract Rheumatol 2: 35-43, 2006). The balance between bone and adipocyte generation in bone marrow is affected by factors including aging, osteoporosis, and activation of PPARγ. The present inventors have shown, using qPCR, that BMSCs isolated from OPN KO mice were significantly more osteogenic and less adipogenic than BMSCs isolated from WT mice which suggests that OPN is an important regulator of both differentiation pathways. A similar bone marrow phenotype was reported in mice expressing a deletion mutant FosB transgene (Kveiborg M, et al., DeltaFosB induces osteosclerosis and decreases adipogenesis by two independent cell-autonomous mechanisms. Mol Cell Biol 24: 2820-2830, 2004). The data herein correlate with the reports that OPN KO mice have greater bone mineralization than WT mice (Harmey D, et al., Elevated skeletal osteopontin levels contribute to the hypophosphatasia phenotype in Akp2(−/−) mice. J Bone Miner Res 21: 1377-1386, 2006) and are resistant to models of bone loss (Ishijima M, et al., Osteopontin is associated with nuclear factor kappaB gene expression during tail-suspension-induced bone loss. Exp Cell Res 312: 3075-3083, 2006.; Yoshitake H, et al., Osteopontin-deficient mice are resistant to ovariectomy-induced bone resorption. Proc Natl Acad Sci USA-96: 8156-8160, 1999).

Adipose tissue-derived stromal cells (ATSCs) and BMSCs have similar gene expression patterns and osteogenic and adipogenic differentiation potentials (Lee R H, et al., Characterization and expression analysis of mesenchymal stem cells from human bone marrow and adipose tissue. Cell Physiol Biochem 14: 311-324, 2004). Thus, the OPN KO ATSCs, like OPN KO BMSCs, may be less adipogenic than WT ATSCs. Little is known about the mechanisms regulating adipocyte hypertrophy. HFD-fed OPN KO mice have decreased adipocyte hypertrophy compared to WT mice, which may be due to decreased ability of the adipocytes to fully differentiate to maturity (reduced hypertrophic capability), and possible compensatory adipocyte hyperplasia to accommodate the high lipid load of HFD. Adipocyte hypertrophy and differentiation require extensive extracellular matrix remodeling (Chun T H, et al., A pericellular collagenase directs the 3-dimensional development of white adipose tissue. Cell 125: 577-591, 2006; Gregoire F M. Adipocyte differentiation: from fibroblast to endocrine cell. Exp Biol Med (Maywood) 226: 997-1002, 2001; Nakajima I, et al., Adipose tissue extracellular matrix: newly organized by adipocytes during differentiation. Differentiation 63: 193-200, 1998). Given the well-characterized role of OPN in extracellular matrix remodeling, OPN deficiency may impair adipocyte hypertrophy/differentiation through dysregulation of extracellular matrix. Notably, the effects of OPN deficiency on in vitro adipogenic BMSC differentiation demonstrate that the role of OPN in insulin resistance and adipocyte biology extends beyond it role in modulating immune cell function.

The present inventors have made the novel observation that two week high fat diet impedes subsequent in vitro BMSC osteogenic differentiation and enhances adipogenic differentiation in WT not in OPN KO mice. In the WT mice, this effect may be mediated by PPARγ ligands, either components or metabolites of the HFD, that have long term effects on the differentiation potential of BMSCs. This finding correlates with in vivo published studies relating a balance between bone marrow adiposity and bone density (Gimble et al., 2006). Interestingly, the high fat diet effect on BMSC differentiation potential was not observed in the OPN KO mice and is further evidence that OPN is a positive regulator of adipogenesis and negative regulator of osteoblastic differentiation. The adipogenic and osteogenic differentiation potential of BMSCs from these mouse groups needs to be explored further using additional markers of differentiation.

In summary, we find that OPN is a key modulator of the early onset of high fat diet-induced insulin resistance in liver, muscle and adipose tissue. We provide evidence that the mechanism by which the OPN KO mice are protected from HFD-induced insulin resistance involves reduced leptin expression, decreased hypertrophy of adipocytes, and suppression of inflammatory cytokine secretion in adipose tissue. OPN KO mice are protected from atherosclerosis in pro-atherosclerotic mouse backgrounds and Bruemmer, et al. have shown that this is mediated by leukocyte-derived OPN (Bruemmer et al., 2003; Matsui Y, et al., Osteopontin deficiency attenuates atherosclerosis in female apolipoprotein E-deficient mice. Arterioscler Thromb Vasc Biol 23: 1029-1034, 2003). Nomiyama et al. have recently demonstrated that OPN deficiency alleviates insulin resistance induced after long term high fat feeding, in part, by reducing macrophage infiltration into adipose tissue (J Clin Invest 117: 2877-2888, 2007). The cell type(s) that mediates the role of OPN in insulin resistance in our two-week HFD model is unclear at this time and is the focus of current studies in our laboratory. OPN is novel participant in the early pathogenesis of diet-induced insulin resistance and insulin target tissue biology. As such, OPN may be an attractive therapeutic target for the treatment of human insulin resistance and type 2 diabetes.

Accordingly, in one embodiment, the present invention provides a method for treating diabetes mellitus and related disorders, such as obesity or hyperglycemia, by administering to a subject an inhibitor of OPN in an amount sufficient to ameliorate the symptoms of the disease. Type 2 or noninsulin-dependent diabetes mellitus (NIDDM), in particular, is characterized by a triad of (1) resistance to insulin action on glucose uptake in peripheral tissues, especially skeletal muscle and adipocytes, (2) impaired insulin action to inhibit hepatic glucose production, and (3) dysregulated insulin secretion (DeFronzo, (1997) Diabetes Rev. 5:177-269). Therefore, subjects suffering from type 2 diabetes can be treated according to the present invention by administration of an OPN inhibitor, which increases sensitivity to insulin and glucose uptake by cells. Similarly, other diseases characterized by insulin dysfunction (e.g., resistance, inactivity or deficiency) and/or insufficient glucose transport into cells also can be treated according to the present invention by administration of an OPN inhibitor, which increases sensitivity to insulin and glucose uptake by cells.

### Materials and Methods

### Example MM1

### Human Studies

Five lean, insulin sensitive and 6 obese, insulin resistant subjects were treated with pioglitazone (45 mg/day) for 3 months. The clinical characteristics of the patients are shown in Table 1. Before and after pioglitazone treatment, a subcutaneous adipose tissue biopsy from each patient was harvested and flash-frozen in liquid nitrogen and each patient was subjected to a 5 hr 60 mU/m2/min hyperinsulinemic-euglycemic clamp. Baseline plasma samples were drawn and hyperinsulinemic-euglycemic clamps were performed in the morning after a 10 hr fast as previously described (Frias, J P et al., 2000, Diabetes Care 23, 64-69; Yu, J G et al., 2002, Diabetes 51, 2968-2974). The experimental protocol was approved by the Institutional Review Board of the University of California, San Diego. Informed written consent was obtained from each subject.

### Example MM2

### Animal Strains

Male C57Bl/6J WT mice (cat #000664) and OPN KO mice (B6.Cg-Spp1tm2blh/J, cat #004936) were purchased from Jackson Laboratories. This OPN KO mouse line has been backcrossed into the C57Bl/6J background >10 generations. Mouse diets were as follows: normal chow diet (12% kcal from fat; Purina 5001, LabDiet) and high fat diet (41% kcal from fat; TD96132, Harlan Teklad). Mice were 4-6 months of age and age-matched in all studies. Male lean Zucker (fa/+) and fatty Zucker (fa/fa) rats were purchased from Charles River. Lean rats were fed normal chow, fatty rats were fed normal chow or normal chow dosed to deliver 10 mg/kg/day pioglitazone for three weeks. All rats were nine weeks of age at time of terminal metabolic studies and tissue harvest. All animals were housed 1-3 per cage under controlled light (12:12 light:dark) and climate conditions. Animals had unlimited access to food and water.

All procedures were performed in accordance with the Guide for Care and Use of Laboratory Animals of the National Institutes of Health and were approved by the University of California, San Diego, Animal Subjects Committee.

### Example MM3

### In Vivo Metabolic Studies in Rats

Insulin sensitivity was determined by hyperinsulinemic euglycemic clamp, as previously published (Hevener, A L, et al., 2001, Diabetes 50, 2316-2322). A variable infusion of glucose (50% dextrose; Abbott Laboratories) was used, along with an infusion of tracer (0.16 μCi/min) and insulin (25 mU/kg/min, Novlin R; Novo Nordisk, Copenhagen). At the end of the clamp procedure, the animals were administered a lethal injection of sodium pentobarbital (100 mg/kg; Nembutal; Abbott Laboratories). Plasma glucose specific activity was measured after deproteinization with barium hydroxide and zinc sulfate (Revers, R R, 1984, J Clin Invest 73, 664-672). Hepatic glucose output (HGO) and glucose disposal rate (GDR) were calculated for the basal period and the steady-state portion of the glucose clamp using the Steele equation for steady-state conditions (Steele R. Influences of glucose loading and of injected insulin on hepatic glucose output. Ann N Y Acad Sci 82: 420-430, 1959). Matched rat groups not subjected to hyperinsulinemic-euglycemic clamp studies were used for adipose tissue analyses. Tissues from these rats were excised after lethal injection, immediately flash-frozen in liquid nitrogen, and stored at −80 T C for subsequent in vitro analyses.

### Example MM4

### In Vivo Metabolic Studies in Mice

Insulin sensitivity was assessed using a submaximal hyperinsulinemic euglycemic glucose clamp technique as previously described (46) with the following modifications: 1) isoflurane was used for anesthesia, 2) glucose tracer was infused at 2 μCi/hr, and 3) insulin was infused at 3 mU/kg/min. The animals were allowed to recover. Four days later, mice were fasted for 5 hr and then anesthetized (isoflurane) to collect blood (cardiac puncture), and then euthanized (pentobarbital) to collect gastrocnemius muscle, liver, epididymal and inguinal fat. Half of each tissue sample was flash-frozen in liquid nitrogen and half was fixed in Zn-formalin. Plasma glucose specific activity, GDR, and HGO were calculated as described above. Acute insulin stimulation was achieved by intraperitoneal injection of 6 hr fasted mice with 0.85 U/kg insulin. After 15 min., tissues were harvested as above.

### Example MM5

### Plasma and Tissue Analyses

Plasma insulins were measured by radioimmune assay kit (Linco Research, St. Charles, Mo.). Plasma FFA levels were measured enzymatically using a commercially available kit (NEFA C; Wako Chemicals USA). Triglycerides were measured using the Triglyceride-SL Assay (Diagnostic Chemicals Ltd.). Cholesterol was measured using the Chol kit and Roche/Hitachi analyzer (Roche). Tissue lysates were analyzed by SDS-PAGE, western blotting and chemiluminescence and by ELISA. Signal intensities of chemiluminescence autoradiographs were densitometrically quantified using a digital Kodak 3D Imagestation and associated digital image analysis software (Kodak, New. Haven, Conn.). IL-1β, IL-12p70, IFNγ, IL-6, IL-10, Cxcl1 and TNFα levels in plasma and tissue lysates were measured using a multiplex (7-plex) ELISA (Meso Scale Discovery).

### Example MM6

### Histological Studies

Excised fat pads were immediately fixed in Zn-formalin overnight, transferred to 70% ethanol, and subsequently paraffin-embedded. Paraffin sections stained with hematoxylin and eosin were used for determining cell size, as previously described (Miles, P D, et al., 2000, J Clin Invest 105, 287-292). All digital images of tissue sections were captured using the same microscope magnification. Microscopic fields with minimal non-adipocyte material were selected for quantitation of cell number per field. There was no apparent difference in non-adipocyte material in the sections between the mouse groups. Three fields were captured per mouse fat pad, from five mice in each group. Section images were visualized and cells per field image counted using ImageJ software (NIH freeware). Adipocyte size is represented by the inverse of the adipocyte number per field. Immunohistochemistry was performed using a Mac-2 antibody (Cedarlane Laboratories, Ltd., Hornby, Ontario, CANADA) to identify macrophages.

### Example MM7

### Isolation and Differentiation of Plastic-Adherent Bone Marrow Stromal Cells (BMSCs)

Femurs from the indicated mouse groups were flushed with 1% FCS-containing DMEM low glucose medium. The washed cells from the femurs were subsequently centrifuged for 10 min. at 500×g and cultured for 14 days in Basal Mesenchymal Stem Cell (MSC) medium (Cambrex, Walkersville, Md.) supplemented with 1% glutamine (w/v), 100 U/ml Penicillin, 50 μg/ml Streptomycin, and 10% FCS. Differentiation of cultured BMSCs was conducted as previously described (Sciaudone, M, et al., 2003, Endocrinology 144, 5631-5639; Sekiya, I, et al., 2004, J Bone Miner Res 19, 256-264) with slight modifications. For adipogenic differentiation, the BMSCs were plated in monolayer in MSC medium with the addition of 0.5 uM dexamethasone, 50 uM indomethacin and 0.5 mM IBMX. The cells were grown for the days indicated and the media was replaced every three days. For osteogenic differentiation, BMSCs were plated in monolayer in αMEM medium containing 10% FCS, 0.5 UM dexamethasone, 50 Ug/ml ascorbic acid, 10 mM β-glycerophosphate and grown as above.

### Example MM8

### RNA Isolation and Quantitation

Total RNA was isolated from human adipose tissue and BMSCs using Trizol (Invitrogen) and from rat adipose using the RNeasy Lipid Tissue Kit (Qiagen). Human and rat adipose RNA quantitation: One step quantitative real-time PCR was carried out on 10 ng human or rat RNA. Primers and probes used were as follows: human OPN: forward, 5′-AGTTTCGCAGACCTGACATCCAGT-3′ SEQ. ID NO. 1; reverse, 5′-TTCATAACTGTCCTTCCCACGGCT-3′ SEQ. ID NO. 2; probe, 5′FAM TGGAAAGCGAGGAGTTGAATGGTGCA-TAMRA-3′ SEQ. ID NO. 3; rat OPN: forward, 5′-TATCAAGGTCATCCCAGTTGCCCA-3′ SEQ. ID NO. 4; reverse, 5′-ATCCAGCTGACTTGACTCATGGCT-3′ SEQ. ID NO. 5; probe, 5′-FAM-TCTGATCAGGACAGCAACGGGAAGA-TAMRA-3′ SEQ. ID NO. 6. Reactions were run on a 7900 Real-Time PCR System (Applied Biosystems) in a final volume of 20 UI containing 400 nM of the forward and reverse primers, 200 nM probe, 1× iScript Reverse Transcriptase and 1× iTaq RTPCR-Master Mix (BioRad). Reactions were performed in triplicate. Cycling parameters were as follows: 50° C. for 10 min. and 95° C. for 5 min., followed by 40 cycles at. 95° C. for 10 sec and 60° C. for 30 sec. Absolute quantitation was achieved by comparing to an OPN standard curve constructed using human or rat Universal Reference RNA standard (Stratagene). The standard curves had r2 values of at least 0.99. Additionally GAPDH expression was used to confirm equal sample loading. BMSC RNA quantitation: RNA isolated from BMSCs was converted into cDNA using reverse-transcriptase and dNTPs. For qPCR, 1 μL of a 25-fold dilution of the cDNA from specific reverse transcription reactions (above) was amplified using the LightCycler FastStart DNA MasterPlus SYBR Green I kit (Roche Diagnostics, Indianapolis, Ind.) with addition of 0.5 μM of each primer in the LightCycler 2.0 (Roche Diagnostics, Indianapolis, Ind.). Following amplification, a monocolor relative quantification of the target gene and reference GAPDH analysis was done to determine the normalized target gene/GAPDH mRNA copy ratios by the manufacturer's LightCycler Software (Version 4.0). The following primers were used: mouse GAPDH: forward 5′-CATCCCAGAGCTGAACG-3′ SEQ. ID NO. 7; reverse 5′-CTGGTCCTCAGTGTAGCC-3′ SEQ. ID NO. 8; mouse OSX: forward 5′-CTCTCTTTGTCAAGAGTCTTAGC-3′ SEQ. ID NO. 9; reverse 5′-AGAAAGATTAGATGGCAACGAGTTA-3′ SEQ. ID NO. 10; mouse PPARγ: forward 5′-AGAGTCTGCTGATCTGCG-3′ SEQ. ID. NO. 11; reverse 5′-TCCCATCATTAAGGAATTCATGTCGTA-3′ SEQ. ID NO. 12; mouse Akap2: forward 5′-AGACACAAGCATTCCCACTAT-3′ SEQ. ID NO. 13; reverse 5′-CACCATCTCGGAGACCG-3′ SEQ. ID NO: 14. All primers were designed using the LightCycler Probe Design Software 2.0.

### Example MM9

### Statistical Analyses

Student's t-test and ANOVA were used for statistical analyses. P-values for correlations were determined using a linear correlation analysis (GraphPad Prism) using 2 tailed Pearson correlation coefficient. A p-value cutoff of 0.05 was used to determine significance after statistical tests.

It is understood that the examples and embodiments described herein are for illustrative purposes only and that various modifications or changes in light thereof will be suggested to persons skilled in the art and are to be included within the spirit and purview of this application. All publications, patents and patent applications cited herein are hereby incorporated by reference in their entirety for all purposes to the same extent as if each individual publication, patent or patent application were specifically and individually indicated to be so incorporated by reference.

It is claimed:

1. A method of increasing insulin sensitivity in a subject having insulin resistance comprising decreasing osteopontin activity in the subject.

2. The method of claim 1, wherein said subject has type II diabetes.

3. The method of claim 1, wherein said osteopontin activity is decreased by administration of an osteopontin inhibitor.

4. The method of claim 3, wherein the inhibitor is selected from the group consisting of an antibody, an antibody fragment, siRNA, and an aptamer.

5. The method of claim 4, wherein the antibody is a monoclonal antibody.

6. The method of claim 4, wherein the antibody is human antibody.

7. The method of claim 4, wherein the antibody is humanized antibody.

8. The method of claim 1 further comprising increasing cellular uptake of glucose in said subject.

9. The method of claim 1 further comprising measuring a decrease in a cytokine in a body fluid of said subject, wherein the cytokine is selected from the group consisting of leptin, IL-1β, IL-12p70, IFN-γ, IL-6, Cxcl1, IL-10, and TNF-α.

10. A method of increasing glucose uptake by cells in a subject comprising administering to the subject an osteopontin inhibitor.

11. The method of claim 10, wherein the cells are adipocytes or a precursor thereof.

12. A method of treating metabolic syndrome in a subject in need thereof comprising administering to the subject an osteopontin inhibitor.

13. The method of claim 12, wherein said metabolic syndrome is diabetes.

14. The method of claim 12, wherein said osteopontin inhibitor is an antibody, antibody fragment, siRNA, or aptamer.

15. The method of claim 12, wherein said antibody is a monoclonal antibody.

16. The method of claim 12, wherein said antibody is a human antibody.

17. The method of claim 12, wherein said antibody is a humanized antibody.

18. The method of claim 12, wherein said antibody is an antibody fragment or derivative.

19. A method of increasing insulin sensitivity and glucose uptake by muscle or hepatocyte cells, comprising administering to muscle or hepatocyte cells, respectively, an osteopontin inhibitor.
